(19) United States
(12) Patent Application Publication
(10) Pub. No.: US 2012/0082677 A1
(43) Pub. Date: Apr. 5, 2012

(54) COMPOSITIONS FOR THE TREATMENT OF HCV AND HBV

(76) Inventor: Sherif Salah Abdul Aziz, Cairo (EG)

(21) Appl. No.: 12/896,564

(22) Filed: Oct. 1, 2010

(51) Int. Cl. A61K 39/40; A61P 31/04; G01N 33/53; A61K 31/195; A61K 38/45

(52) U.S. Cl. 424/150.1; 514/561; 424/165.1; 424/94.5; 436/501

(57) ABSTRACT

Therapeutic compositions for the treatment of viral hepatitis B (HBV) and hepatitis C (HCV) comprise antistreptokinase and antistreptolysine antibodies and/or tranexamic acid. Three antibodies against: polymerase, plasmin activators and fibrin are found in the blood of hepatitis C infected patients.

### TECHNICAL FIELD

This invention relates to the presence of antibodies in a subject having a hepatitis infection and therapeutic compositions for the treatment of viral hepatitis B (HBV) and hepatitis C (HCV). In particular the invention relates to a composition of antibodies having an anti HCV and HBV effect. The present invention also relates to the presence of three antibodies against: polymerase, plasmin activators and fibrin in the blood of hepatitis C infection.

### BACKGROUND

Hepatitis C virus (HCV), a member of the Flaviviridae family, is a major cause of chronic hepatitis, liver cirrhosis and hepatocellular carcinoma worldwide. HCV is an enveloped virus with a positive single-stranded RNA genome that encodes a single polypeptide precursor of about 3000 amino acids that is cleaved into 10 proteins by cellular or viral proteases These proteins include three structural proteins, one core (C) protein and two envelope (E1 and E2) glycoproteins, and seven non-structural proteins (NS1-NS5B) involved with polypeptide processing and RNA replication.

There are a number of treatment options for viral hepatitis. Combined treatment with pegylated interferon (IFN) and ribavirin is the current standard treatment for HCV and HBV infection.

Oligonucleotides which destroy or incapacitate HCV RNA in the cytoplasm to prevent or to reduce attachment of the RNA to the endoplasmic reticulum and to impair translation of the large open reading frame (ORF) that produces the HCV polypeptide, have been proposed to treat HCV. Protease inhibitors VX-950 and SCH 503034 have recently entered clinical trials. Interluekin-2, interleukin-12, immunomodulatory agents such as thymosin and levamisole, herbal therapy using Phyllanthus amarus, and nucleoside analogs such as lamivudine and famcyclovir have been also been proposed as treatment options for viral hepatitis.

However these options are not always successful and can have a large number of side effects. With interferon therapy the majority of patients develop significant adverse symptoms such as flu-like symptoms, fatigue, headaches, alopecia, nausea, abdominal pain, thrombocytopenia and the production of autoantibodies. Also cytokines such as interleukins, also being involved in the enhancing hormone activity, can also produce similar side effects to interferon. In oligonucleotide therapy, problems arise due to the potential for drug inactivation by host nucleases prior to the drug reaching its target site. The inhibition of the HCV NS3/NS4A protease has proved difficult since the protease binding groove is shallow and the affinity for bound substrates is weak. Until recently vaccines have not succeeded because the virus has a great ability to change itself via its large open reading frame.

Therefore the object of the invention is to provide an alternative treatment which provides antibodies to allow the immune cells to be highly expressed in acute and chronic hepatitis patients.

### SUMMARY

This invention provides a pharmaceutical composition for the treatment of hepatitis B or hepatitis C comprising tranexamic acid. The tranexamic can help in preventing fibronolysis of fibrin.

The pharmaceutical composition can further comprise an antistreptolysin antibody and for an antistreptokinase antibody.

The pharmaceutical composition can further comprise a DNA pfu polymerase enzyme of bacterial origin. The antibodies suppress plasminolysis activity of the hepatitis virus and stabilize the fibrin in blood circulation.

A second embodiment of the invention comprises a pharmaceutical composition for the treatment of hepatitis B or hepatitis C comprising an antistreptolysine antibody and an antistreptokinase antibody.

A third embodiment of the invention provides a pharmaceutical composition for the treatment of hepatitis B or hepatitis C comprising DNA pfu polymerase enzyme of bacterial origin.

The pharmaceutical composition can further comprise an antistreptolysine antibody.

The antibodies are human antibodies. The antibodies are monoclonal antibodies or are polyclonal antibodies.

Methods of treating hepatitis B or hepatitis C comprise administering the pharmaceutical compositions to a patient. The compositions can be administrated subcutaneously or intramuscularly.

An embodiment of the invention comprises a method for detecting hepatitis C in a patient comprising determining the presence of an anti-plasmin antibody, an antifibrin antibody and an anti-polymerase antibody in a serum sample of the patient.

An embodiment of the invention comprises a method for detecting hepatitis in a patient comprising determining the presence of tissue plasminogen activators inhibitors antibodies and tissue plasmin activators antibodies in the blood of the patient.

The tissue plasminogen activator inhibitors can be TPI or TPII.

### DETAIL DESCRIPTION

The new therapeutic compositions are based on two types of antibodies, anti streptolysine antibodies and antistreptokinase antibodies for subcutaneous or intramuscular administration. Antistreptolysine antibodies and anti streptokinase antibodies are secreted by the body's immune cells as a defense mechanism when the body is infected by the bacteria Streptococcus haemolyticus. The antibodies have the ability to stop the action of plasmin antigen. Antistreptolysin and antistreptokinase antibodies in combination with any drug having an anti fibrinolytic action, such as tranexamic acid, a synthetic amino acid, has a great ability to enhance the enzyme activity of plasmin inhibitors and neutralize the antibodies against plasmin inhibitors.

The hypothesis depends on any biological processes requiring a balance between the proteases that initiate the proteolysis pathways essential to life and the inhibitors that limit excessive protease activity. Coagulation and fibrinolysis, are the two processes that form and dissolve fibrin, respectively. These two processes are regulated and protect the body from excessive blood loss or excessive fibrin deposition and degradation. We hypothesize that hepatitis patients have a defect in the inhibitors mechanism, which play an important role in activating the consciousness of the immune cells.

If HCV succeeds in:
1—stimulating the plasminogen receptors in fibrinogen and exerts oxidative stress on immune cells to produce antibodies against antiplasmin inhibitors; and
2—the process of replication continues to takes all the polymerase enzyme of the cell;
   the HCV suppress the immune cells for producing anti-polymerase against the polymerase so the processes of fibrinolysis and replication will continue without stoppage.

We detected three antibodies in HCV patients and obtained good evidence for the presence of the three antibodies in serum of infected patients compared to normal control persons.

The liver of the infected person contains fibrin. Fibrin is made from a zymogene fibrinogen, a soluble plasma glycoprotein, which is synthesized by the liver. Processes in the coagulation cascade activate the zymogene prothrombin to become the serine protease thrombin, which is responsible for converting fibrinogen into fibrin. Fibrin is then cross linked by factor XIII to form a clot. Fibrinogen is a 340 kDa glycoprotein synthesized in the liver hepatocytes and megakaryocyte, which normally has a concentration between 1.5-4.0 g/L (measured using the Clauss method) in blood plasma. In its natural form, fibrinogen is useful in forming bridges between platelets, by binding to their GpIIb/IIIa surface membrane proteins, though fibrinogen's major use is as a precursor to fibrin in the normal liver.

Sinusoids are formed in a fenestrated endothelium, which is separated from hepatocyte by a sub-endothelial space. Within the sub endothelial space lie hepatic satellite cells, which are quiescent in normal liver. In the injured liver, satellite cells activate into proliferating myofibroblasts that produce scar, while at the same time releasing (tissue inhibitor of metalloproteinases-I) TIMP-I which binds and inactivate the metalloproteinases needed for scar degradation. Thus scar accumulates through increased production and decreased degradation.

For example the low plasma alpha-antiplasmin aggregates in the endoplasmic reticulum of hepatocytes as inclusions at their site of synthesis. In the endoplasmic reticulum, accumulation of the antiplasmin reflects the severity of liver cirrhosis, that means the viruses acts to stimulate the antibodies against the units of serine protease, like stimulation of anti-plasmin antibodies, anti-polymerase and fibrin antibodies.

In polymerase chain reaction we depend on the active memory of the polymerase enzyme, which has the power to make millions of copies for the RNA strand in short time, according to the type of polymerase enzyme and which type of bacteria the enzyme originated. This can effect the results of the PCR count, as some types of bacteria have the ability to stop shortly to make order and the other may extended the processing the same order. This makes a difference in consuming the polymerase of the cell, the same thing happens in the infected persons for HCV and the cell persists for a time in replication to form multi signals without stoppage.

This is the difference between the experienced cells and the non experienced ones, because the former has auto antibodies (Anti polymerase) which prevent it from correct cooperation. So if we can stimulate the immune cells gradually to produce anti polymerase we shall give the cell the chance to be educated faster than the previous cells with HCV infection.

DNA pfu polymerase I is the most active memory units within our cells. This enzyme functions in bacterial cells to repair DNA, while the DNA pfu polymerase is responsible for replicating the genome with higher proof reading There are several additional DNA pfu polymerase in bacterial cells of which the precise function is not known. Some of these enzymes might be necessary to replicate genomic DNA that has been damaged.

This enzyme is regulated by mechanism, including serine inhibitors and proteolysis. If you have an inhibitor mechanism that prevents excessive utilization of polymerase the cell will continue to produce the same answer for a long time to keep the cell in one state. While if you add it with other monoclonal antibodies you shall educate the immune cells to form anti polymerase and the cells by this mechanism will go and return back shortly.

We started trials to prove the presence of the two antibodies in viral infected hepatitis patients and started trials to use a mechanism that acts against the antibodies to eradicate the virus.

1—We searched for anti-plasmin antibodies in samples of patients infected by HCV RNA.
2—We searched for anti-polymerase antibodies in samples of patients infected by HCV RNA.
3—We searched for anti-fibrin antibodies in samples of patients infected by HCV RNA

To test the hypothesis that there are anti-plasmin antibodies and anti-polymerase antibodies, and anti-fibrin antibodies in hepatitis HCV infected patients we firstly examined infected patients.

### Experiment 1

Participants: 34 hepatitis patients and 10 normal control subjects were examined. All samples from participants were analyzed.

### Results

We determined the ratio between the quantities measured of HCV RNA, the amount of anti-polymerase antibody, anti-plasmin antibody and anti-fibrin antibody and the level of liver enzymes were also measured.

A high viremia value for HCV RNA by PCR correlates with low anti-polymerase antibody level, anti plasmin antibody level and high level for anti fibrin antibody levels and with a slight elevation of liver enzymes of about one to two folds higher than normal values

A low to very low viremia value for HCV RNA by PCR we found high titer for anti-polymerase antibodies, anti-plasmin and low anti fibrin antibodies with marked elevation of liver enzymes of three to five fold higher than normal.

In control patients we found three cases with a slight increase in titer of antiplasmin with no viremia and no anti-polymerase antibody. Anti-plasmin, anti-polymerase and anti fibrin antibodies levels in hepatitis patients are completely difference higher than in normal control subjects.

### Experiment 2

This experiment was carried out on 100 HCV infection patients which included 78 males and 22 females and 33 HBV infection patients, all of them males. The ages ranged from 30-60 years. The patients had already been previously diagnosed as HCV or HBV infected patients and was confirmed by PCR.

HCV-PCR and HBV PCR was done before and after each therapeutic trial. 77% of all studied HCV infected patients and 85% of all HBV infected patients had undetectable level using HCV-PCR and HBV-PCR quantitatively.

The remaining cases still showed positive with variable results, using the same method, but at a lower level than in the beginning of the study. From this work it can be believed that the current therapeutic trial has an effect on HCV and HBV infection.

### 6 Months Follow Up:

All studied cases (77) were reexamined after 6 months. Sixty patients returned for this follow up while the remaining 17 patients did not come. All 33 patients of the HBV cases were followed up six months later. Only 18 of the studied cases returned for follow up and the remaining (15) cases did not come

Using HCV-PCR and HBV-PCR analysis 57 people were still within an undetectable level of HCV and HBC. While the remaining three patients showed lower viremia which may be due to relapse or due to reinfection after asking patient if he was exposed to infected blood or products.

For the HBV infected patient, using HBV-PCR all 18 of the follow up cases showed undetectable levels of HBV.

We have good prognosis for patients with clinical data improvement in biochemical data like liver functions (enzymes synthetic functions) complete changes in hematological data especially number of blood platelets and leukocyte count and increasing in the titer value of HCV antibodies titer as immunological data. Results are summarized in Table 1 and 2.

TABLE 1
Response of 100 studied cases for therapeutic trials

HCV infected
Responded cases to therapeutic trails

| Case No of | | | % to total | |
|---|---|---|---|---|
| Therap. Trail | NO | % | studied cases | |
| 1^{st} Trial | 30/100 | 30% | 30% | 70 |
| 2^{nd} Trial | 37/100 | 52.86% | 37% | 33 |
| 3^{rd} Trial | 9/100 | 27.3% | 9% | 24 |
| 4^{th} Trial | 1/100 | 4.17% | 1% | 23 |
| Total | 77/100 | | 77% | 23/100 |

TABLE 2
Follow up after (6) months for all studied
(77) cases with (Undetectable level)

| Studied cases | No. of cases |
|---|---|
| Cases still with undetectable level: | 57 |
| Cases with lower level; | 3 |
| Relapse | 1 |
| Re infection | 2(Exposed to infected |
| | blood material) |
| Cases don't complete the follow up | 13 |

### Materials Provided:

1—Chemical component—transexamic acid (1^{st} component—a drug used in therapy)
2—Non specific polyclonal antibodies of human source—anti streptolysine antibodies; antistreptokinase antibodies (2^{nd} component).

### Composition and Standardization:

A mixture is formed of the 2 components 100 mg/ml under complete aseptic conditions by mixing 1 volume of the first component with 1 volume of second component.

The resulting preparation was tested for; HIV, HIV2, HCV and HBV. The resulting mixing solution was sterilized by filtration and carefully put in a sterilized ampoule sealed after this. The mixture was stored +2 to +8 C.°.

### Procedure:

The ampoule containing the mixture is brought to room temperature (+15 C.° to +25 C.°. The mixture is mixed well immediately prior to use.

Injection was done gradually according to patient weight as following:

1^{st} dose; 15 cc ampoule of the mixture; one hour after light meal (2 times daily with 2 hours interval)

Seven days rest for patient before having the 2^{nd} dose.

2^{nd} ampoule but with increasing the dose up to 1.5 cc (2 times daily with 2 hours interval)
3^{rd} dose; 7 days rest before starting the 3^{rd} ampoule in the same manner mentioned in 2^{nd} dose
4^{th} dose; 7 days rest before starting the 4^{th} ampoule in the same manner as above but increasing the dose to 2 cc (2 times daily with 2 hours interval)

### Evaluation:

Rest 20 days after which HCV-PCR and HBV-PCR was performed as well as other investigations done before the trial began.

### Dosage

The composition is given intramuscularly

1^{st} dose trial 15 cc daily dose, 2 cc IM
2^{nd} dose trial 15 cc daily dose, 3 cc IM
3^{rd} dose trial 15 cc daily dose, 3 cc IM
4^{th} dose trial 15 cc daily dose, 4 cc IM

### Pharmacokinetics of the Trial:

The chemical part is easily absorbed via the GIT with peak plasma concentration occurring after 2 hours with very low protein binding. It is excreted in the urine mainly as unchanged drug. For the antibodies renal absorption occurs as for any protein molecules. It is excreted in the bile, urine and saliva.

### Adverse Reactions:

The mixture appears to be well tolerated with no toxic side effect

### Experiment 3

Experimental animals: We classified 15 white mice in 3 groups every group has 5 mice. Every component of the mixture is injected separately in 1st and 2nd group while the 3rd group is injected with the mixture of the two components for about 15 days for all groups. No side effects or toxic effects were detected and the mice were still alive.

### Experiment 4

In vitro experiment: This therapeutic trial was performed with infected packet of Blood Bank blood, infected HCV blood of moderate viremia patients and infected HBV of donor. By PCR the virus can not be detected in about 10 samples (undetectable levels) while other samples show decreasing the viremia to very mild samples.

### Experiment 5

We examined 15 patients infected by HCV, 11 patients, and HBV, 4 patients, for the action of the component of the mixture separately.

The first five patients (3 HCV patients) and (2 HBV patients) were injected with the drug (first component) for 10 days by I/M injections for 2 cc two times two hours interval.

The second 5 patients (5 HCV patients) injected by the monoclonal antibodies I/M for 1 cc two times two hours interval.

The third group (3 HCV patients and 2 HBV patients) injected with the trial mixture firstly before we reach the final equation for the mixture.

### Experiment 6

Plasma samples were obtained from 25 patients with HCV and 10 normal controls. Medical charts and laboratory test reports for each patient entered in the study were reviewed. ELISA for antibodies against anti plasmin and anti polymerase antibodies showed positive results for all of the infected patients. The control subjects showed the absence of anti-plasmin and anti-polymerase antibodies.

### Experiment 7

Materials provided:
1—Chemical component—Transexamic acid (Kapron-cyclokapron)
(1^{st} component used in therapy as an antifibrinolytic)
2—Non specific monoclonal antibodies of human source (2^{nd} component) (Anti-streptolysine antibodies; anti-streptokinase antibodies)
(Obtained from Vidal, France and Equitech-Bio, Usa)
3—DNA pfu polymerase of bacterial origin

### Subjects:

All patients were subjected to the following:

i. Full history taking
ii. Liver function tests, CBC, Kidney functions tests
iii. For therapeutic trials were done
iv. HCV-PCR and HBV-PCR was performed before and after each

### Composition and Standardization:

Pharmaceutical compositions comprise for every 1 cc:

a) 8000 IU of Antistreptolysin antibodies
b) 12000 IU of Antistreptokinase antibodies
c) 100 mg/ml of Tranexamic acid (a synthetic amino acid).
d) 5 units of PFU polymerase enzyme

A mixture is formed of the 4 components under complete aseptic conditions by mixing 1 volume of a component, comprising 8000 IU of antistreptolysin antibodies+12000 IU of antistreptokinase antibodies with 1 volume of second component, comprising 100 mg/ml of transexamic acid, and 5 units of PFU polymerase.

The resulting preparation was tested for; HIV, HIV2, HCV and HBV. The resulting mixing solution was sterilized by filtration and carefully put in a sterilized ampoule sealed after this. The mixture was stored +2 to +8 C.°

### Procedure:

The ampoule containing the mixture is brought to room temperature (+15 C.° to +25 C.°) before injection of 1 ml to the patient. The mixture is mixed well immediately prior to use.

### Pharmacokinetics of the Trial:

The chemical part is easily absorbed via the GIT with peak plasma concentration occurring after 2 hours with very low protein binding. It is excreted in the urine mainly as unchanged drug. For the antibodies renal absorption occurs as for any protein molecules. It is excreted in the bile, urine and saliva.

### Adverse Reactions:

The mixture appears to be well tolerated
No toxic side effect

Injection was done; gradually according to patient weight as follows:

1^{st} Dose; 15 cc ampoule of the mixture; one hour after light meal in the first day 1 cc follow by 1 cc (2 times daily with 6 hours interval) two days rest for patient before starting the 2^{nd} dose
2^{nd} dose; 2^{nd} ampoule but with increasing the dose up to 1.5 cc (2 times daily with 6 hours interval)
3^{rd} dose; 2 days rest to start the 3^{rd} ampoule in the same manner mentioned in 2^{nd} dose
4^{th} dose; 2 days rest to start the 4^{th} one (2 times daily with 6 hours interval)

### Evaluation:

Rest 30 days after which; HCV-PCR and HBV-PCR was performed as well as repeating the other investigations done before these trials began.

### CONCLUSION

This is a new approach for therapeutic trials for HCV & HBV infected patients and showed good results without any complications.

### 6 Months Follow Up:

All studied cases (77) of the therapeutic trials were reexamined after 6 months. Sixty patients came for this follow up while the remaining 17 patients did not come.

Using HCV-PCR and HBV-PCR analysis 57 people were still within an undetectable level of HCV and HBC.

While the remaining three patients showed lower viremia which may be due to relapse or due to reinfection after asking patient about if he exposed to infected blood or products. Results are shown in Tables 3 and 4.

Good prognosis for patient's clinical data was obtained, with improvements in biochemical data such as liver functions (enzymes, synthetics functions), complete changes in hematological data especially number of blood platelets and leukocyte count.

TABLE 1
Response of 100 studied cases for therapeutic trails

HCV infected
Responded cases to therapeutic trails

| Case No of | | | % to total | |
|---|---|---|---|---|
| Therap. Trail | No. | % | studied cases | |
| 1^{st} Trial | 30/100 | 30% | 30% | 70 |
| 2^{nd} Trial | 37/100 | 52.86% | 37% | 33 |
| 3^{rd} Trial | 9/100 | 27.3% | 9% | 24 |
| 4^{th} Trial | 1/100 | 4.17% | 1% | 23 |
| Total | 77/100 | | 77% | 23/100 |

TABLE 2
Follow up after (6) months for all studied
(77) cases with (Undetectable level)

| Studied cases | No. of cases |
|---|---|
| Cases still with undetectable level: | 57 |
| Cases with lower level; | 3 |
| Relapse | 1 |
| Re infection | 2(Exposed to infected |
| | blood material) |
| Cases don't complete the follow up | 13 |

### Experiment 7

Experimental animals: We classified 20 white mice in 4 groups every group has 5 mice. Every component of the mixture (antistreptolysin antibodies; antistreptokinase antibodies; and tranexamic acid) is injected separately in the 1st, 2nd and 3rd groups while the 4th group is injected with the mixture of the three components, for about 15 days for all group. No side effects or toxic effects were detected and the mice were still alive.

1. A pharmaceutical composition for the treatment of hepatitis B or hepatitis C comprising tranexamic acid.

2. The pharmaceutical composition according to claim 1 further comprising an antistreptolysin antibody.

3. The pharmaceutical composition according to claim 1 further comprising an antistreptokinase antibody.

4. The pharmaceutical composition according to claim 3 further comprising an antistreptolysin antibody.

5. The pharmaceutical composition according to claim 4 further comprising a DNA pfu polymerase enzyme of bacterial origin.

6. A pharmaceutical composition for the treatment of hepatitis B or hepatitis C comprising an antistreptolysine antibody and an antistreptokinase antibody.

7. A pharmaceutical composition for the treatment of hepatitis B or hepatitis C comprising DNA pfu polymerase enzyme of bacterial origin.

8. The pharmaceutical composition according to claim 7 further comprising an antistreptolysine antibody.

9. The pharmaceutical composition according to claim 6 wherein the antibodies are human antibodies.

10. The pharmaceutical composition as claimed in claim 6 wherein the antibodies are monoclonal antibodies.

11. The pharmaceutical composition as claimed in claim 6 wherein the antibodies are polyclonal antibodies.

12. A method for detecting hepatitis C in a patient comprising detecting the presence of an antiplasmin antibody, an antifibrin antibody and an antipolymerase antibody in a serum sample of the patient.

13. The method according to claim 12 further comprising detecting the presence of tissue plasminogen activator inhibitors TPI or TPII in the blood of the patient.
